(19) United States
(12) Patent Application Publication
(10) Pub. No.: US 2025/0064718 A1
(43) Pub. Date: Feb. 27, 2025

(54) TOPICAL SKIN BALM FOR ANTI-AGING AND HEALING PURPOSES

(71) Applicant: Jordan Nicholas Barnes, Puyallup, WA (US)

(72) Inventor: Jordan Nicholas Barnes, Puyallup, WA (US)

(21) Appl. No.: 18/810,714

(22) Filed: Aug. 21, 2024

Related U.S. Application Data
(60) Provisional application No. 63/520,935, filed on Aug. 22, 2023.

(51) Int. Cl. A61K 8/98; A61K 8/49; A61Q 19/08

(52) U.S. Cl. CPC A61K 8/981; A61K 8/49; A61Q 19/08

(57) ABSTRACT

A topical skin composition for reducing effects of aging and promoting overall skin health may include rendered bone marrow and a methylene blue solution. The composition may also include rendered tallow and a carrier oil. The composition may optionally include an essential oil, an emulsifier, a preservative, a vitamin, a nutrient, a collagen ferrying agent, a collagen ferrying enzyme, and/or a skin opening dilator.

### RELATED APPLICATION

This application claims priority to provisional patent application U.S. Ser. No. 63/520,935 filed on Aug. 22, 2023, the entire contents of which is herein incorporated by reference.

### BACKGROUND

The embodiments herein relate generally to skin care and cosmetics, and more particularly, to a topical skin composition for addressing the effects of aging, improving skin health, aiding in wound healing, and generally helping skin restore itself to homeostasis from a host of ailments.

Human skin faces constant endogenous and exogenous insults-specifically production of reactive oxygen species (ROS) that damage skin cells and ultraviolet radiation from the environment, which compounds the production of ROS. Additionally, ROS inhibit cellular repair mechanisms and propagate further damage to dermal and epithelial cells, which has the overall effect of accelerating skin aging. This effect is compounded further by common skin conditions, such as eczema, psoriasis, general dermatitis, and hypersensitivities, which have the effect of damaging skin due to inflammation, excessive drying, and depletion of the skin's natural oils.

Collagen is the most abundant structural protein in animals and humans. Structural proteins comprise the structure or framework of cells and tissues, such as skin. As people age, the function of fibroblasts, which produce collagen in the skin, tends to become impaired, causing collagen to be fragmented and improperly distributed in the skin. Collagen production can also be negatively impacted by oxidative stress from UV rays in sun exposure as well as other environmental factors and lifestyle choices. The resulting effect is the physical appearance of dermal aging (sagging skin, discoloration, wrinkles, etc.) as people grow older.

The ability of skin to repair itself diminishes over time due to aging and environmental factors, such as UV exposure. Skin is a highly regenerative organ and has a high rate of constant cellular turnover. Keratinocytes, or skin cells, are constantly dividing and producing new skin cells. Keratinocytes need energy to produce new skin cells and aid in repair from environmental damage, such as UV radiation. Cellular energy is produced by the mitochondria, which are often permanently damaged by UV radiation and ROS.

In short, many skin ailments can be traced back to improper mitochondrial function, which provides the energy necessary for proper skin function. Mitochondrial function can be impacted by many different factors.

Therefore, what is needed is a topical skin composition that enhances collagen production, stimulates cellular function, mitigates oxidative stress, and provides a range of skin-enhancing properties to provide anti-aging benefits and to support skin healing.

### SUMMARY

Some embodiments of the present disclosure include a topical skin composition for reducing effects of aging and promoting overall skin health may include rendered bone marrow and a methylene blue solution. The composition may also include rendered tallow and a carrier oil. The composition may optionally include a botanical infusion, an essential oil, an emulsifier, a preservative, a vitamin, a nutrient, a collagen ferrying agent, a collagen ferrying enzyme, and/or a skin opening dilator.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

The composition of the present disclosure may be used to reduce the effects of skin aging and promote skin health and may comprise the following elements. This list of possible constituent elements is intended to be exemplary only, and it is not intended that this list be used to limit the composition of the present application to just these elements. Persons having ordinary skill in the art relevant to the present disclosure may understand there to be equivalent elements that may be substituted within the present disclosure without changing the essential function or operation of the composition.

The various elements of the present disclosure may be related in the following exemplary fashion. It is not intended to limit the scope or nature of the relationships between the various elements, and the following examples are presented as illustrative examples only.

By way of example, some embodiments of the present disclosure include a topical skin composition for reducing effects of aging and promoting overall skin health, the composition comprising rendered bone marrow and a methylene blue solution. In embodiments, the composition may further comprise carrier oil, rendered tallow, essential oils, emulsifiers, herbal infusions, vitamins, nutrients, and preservatives. In fact, various optional additives may be incorporated into the formulation for achieving desired results.

In embodiments, the amounts of the ingredients may vary. However, in a particular embodiment, the composition may comprise 1 part bone marrow, 3 parts tallow, and 1 part carrier oil blend. In some embodiments, the composition may include from about 30 to about 50 volume percent (vol. %), such as about 40 vol. %, bone marrow; about 0.5 to about 3 vol. %, such as about 1 vol. %, methylene blue; about 15 to about 25 vol. %, such as about 19 vol. %, jojoba oil; and a dried botanical infused directly into the marrow/tallow/oil mixture for scent, which adds a negligible weight or volume. In a yet further embodiment, the composition may include a partial bone marrow solution. For example, the composition may comprise about 80 vol. % bone marrow and 20% carrier oil.

In embodiments, the rendered bone marrow may be rendered bone marrow obtained from animal bones from a suitable source, such as beef, bison, porcine, and the like. Bone marrow may be rendered slowly over very low heat, such as from about 100 to about 175° F. Bovine bone marrow is composed mostly of fat and, as such, can be made into skin cream in a manner similar to that of making mallow. Bone marrow include adiponectin, which helps break down facts and maintain insulin sensitivity. Bone marrow also includes glucosamine, glycine, and conjugated linoleic acid, each of which have anti-inflammatory properties. Bone marrow further includes riboflavin, which provides dermatologic manifestations, iron, vitamin E, thiamine (B1), vitamin B12, and vitamin A, each of which provide various health benefits. Bovine bone marrow also includes collagen and stem cells. Thus, when a composition including bone marrow is applied topically, it readily absorbs into the skin, providing a mix of fat-soluble vitamins that closely resembles human skin's sebum. This provides supplemental structural and fat-soluble building blocks for the skin to use in dermal repair.

The methylene blue solution may be a diluted or concentrated solution of methylene blue. Methylene blue may be in the form of methylene blue tincture, methylene blue powder, or methylene blue blocks. Methylene blue has various reported benefits when applied topically: antioxidative properties, anti-inflammatory properties, wound healing properties, collagen stimulation, microbial control, blood circulation, photoprotection, skin brightening, and the like. More specifically, however, methylene blue has three main benefits when applied topically to the skin: (1) mitigation of oxidative stress within cells by being an antioxidative agent for restoring function to fibroblasts to upregulate production of elastin and collagen; (2) blocking of UV radiation and mitigating DNA double-strand breaks caused by UVB radiation in human keratinocytes; and (3) facilitation of wound healing by promoting fibroblast mitigation and proliferation while reducing necrosis progression by blocking nitric oxide and simultaneously providing antimicrobial properties to combat foreign agents. Moreover, methylene blue also provides antimicrobial properties. In fact, a user may forgo the use of soap when washing the face when using the topical composition of the present disclosure. Foregoing the use of soap may help a lot with conditions that result from oil imbalances, such as acne. The use of methylene blue may also abate other conditions that result from bacterial or fungal infections. Lastly, methylene blue may provide helpful benefits in dressing wounds or things like shaving bumps, styes, ingrown hairs, athletes foot, and the like.

The combination of bone marrow and methylene blue may provide a synergistic effect in aiding in skin production, healing, and physiologic functions on a cellular level by reducing cellular oxidative damage and improving mitochondrial function, which in turn improves the utilization of collagen and stem cells provided by bone marrow. Additionally, the inclusion of bone marrow may aid in the dilution of methylene blue, allowing it to be more evenly distributed. Moreover, as bone marrow balm is easily absorbed into the skin, it may aid in the absorption of methylene blue into the dermis. As such, the bone marrow and methylene blue may work synergistically to improve the ability of the skin to use its own energy and constituents provided within the topical cream for skin strengthening and repair, while also improving the skin's innate production of energy and elasticity/collagen content to provide protection against further damage.

As mentioned above, the topical skin composition of the present disclosure may further comprise a carrier oil, rendered tallow, essential oils, emulsifiers, herbal infusions, vitamins, nutrients, and preservatives. Carrier oils may be infused with herbs and combined with the essential oils to provide the composition's aromatic qualities and improve the texture/moisturizing qualities of the composition. Exemplary suitable carrier oils include olive, avocado, jojoba, squalene, coconut, and the like. Exemplary herbal infusions include dried herbs, such as lavender and calendula, infused into carrier oils or directly into rendered fat. Rendered tallow may be included as a base and may serve as the primary fatty component of the composition, contributing to its texture and moisturizing properties. Tallow may be rendered slowly over very low heat, such as from about 100 to 175° F. When included, emulsifiers may help bind the oil-based and water-based components, ensuring a uniform mixture. Exemplary suitable emulsifiers include natural emulsifiers, such as beeswax and lecithin. Optional vitamins or nutrients, such as vitamin E, may be included to provide additional skin nourishment. Preservatives, such as natural preservatives, may be included to extend the shelf life of the composition, helping maintaining its effectiveness over time. Suitable exemplary preservatives include rosemary extract. In alternate embodiments, artificial preservatives may also be used.

In embodiments, a collagen ferrying agent/enzyme or skin opening dilator may also be added to more efficiently incorporate collagen absorption. Exemplary ferrying agents may include naturally occurring hyaluronic acid from plant or animal sources or the enzyme a-1,4-glycosidic hydrolase engineered with genetically encoded 3,4-dihydroxyl-L-phenylalanine. In yet further embodiments, additional ingredients, such as squalene, may be added to make the composition even more closely resemble the natural oils found in human or animal skin.

In some embodiments, the topical skin composition of the present disclosure may be made more effective through a combination therapy process including exposing the skin to red light therapy after the composition has been applied to the skin. In other embodiments, the application of the topical skin composition may be coupled with micro-needling for higher levels of collagen/nutrient absorption and methylene blue proliferation.

Creating the topical skin composition of the present disclosure may comprise preparing the ingredients, which may including collecting and measuring the ingredients to the desired amounts; rendering the tallow and bone marrow, wherein the bone fat and marrow may be filtered, cooled, and repeated until refined down to pure fat with almost no odor; optionally infusing the marrow/tallow/carrier oils with dried herbs by heating and steeping, and then filtering the oil out; preparing the methylene blue solution; optionally melting emulsifiers for proper incorporation; blending the warm ingredients, which may comprise, in a double boiler, slowly stirring while mixing in rendered tallow, rendered bone marrow, infused carrier oils, and any additional additives in the appropriate or desired ratios and continuing to gently heat the mixture to ensure thorough blending and homogenization; initially cooling the mixture by pouring the blended warm solution into a container, such as a bowl, and refrigerating until the mixture begins to solidify; blending the cold ingredients by removing the partially solidified viscous solution from refrigeration, mixing the solution with a mixer, such as an egg beater, kitchen mixer, or the like, and gradually adding the methylene blue solution to the mixture while mechanically agitating the mixture, adding essential oils if desired, and continuing to mix until an even blend is achieved; optionally, whipping the mixture to the consistency of whipped butter; transferring the cooled balm mixture into containers, such as jars or tins; and cooling and solidifying the containers to allow the final product to solidify.

In embodiments, rendering the bone marrow and tallow may including rendering the fats at low heat, purifying by wet rendering with salt, straining, cooling, scraping impurities, and repeating the process until there are no visible impurities, such as about 2 to 3 times. The rendered fat may serve as the base component for the topical skin composition.

To use the topical skin composition of the present disclosure, a user may first cleanse the skin using water or a preferred cleaner and then pat the skin dry. The user may then apply the composition. For anti-aging and daily moisturizer purposes, a small amount of the composition of the present disclosure may be applied to the face, neck, and other target areas using clean fingertips. A gentle upward and outward motion may be used to massage the cream into the skin. The delicate eye area should be avoided. For rashes/injuries/skin irritation purposes, a small amount of the composition may be applied to the affected area using clean fingertips and a gentle circular motion until the composition is fully absorbed. After application, the skin may be massaged to help improve circulation, aid absorption, and promote a relaxing experience. The composition should be allowed to be completely absorbed before the application of any additional products. For best results, the composition may be used twice daily-once in the morning and once at night. Consistency of use may provide improved results over time.

While the composition is described above as being a standalone product, it is envisioned that it may be incorporated into other topical products, such as cosmetics, wound care products, and the like.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

What is claimed is:

1. A topical skin composition for reducing effects of aging and promoting overall skin health, the composition comprising:
rendered bone marrow; and
a methylene blue solution.

2. The composition of claim 1, further comprising a carrier oil.

3. The composition of claim 1, further comprising rendered tallow.

4. The composition of claim 1, further comprising a member selected from the group consisting of an essential oil and a botanical infusion.

5. The composition of claim 1, further comprising an emulsifier.

6. The composition of claim 1, further comprising a member selected from the group consisting of a vitamin and a nutrient.

7. The composition of claim 1, further comprising a preservative.

8. The composition of claim 1, further comprising a member selected from the group consisting of a collagen ferrying agent, a collagen ferrying enzyme, and a skin opening dilator.

9. A topical skin composition for reducing effects of aging and promoting overall skin health, the composition comprising:
rendered bone marrow;
a methylene blue solution;
rendered tallow; and
a carrier oil.

10. The composition of claim 9, further comprising a botanical infusion.

11. The composition of claim 10, wherein:
the carrier oil is jojoba oil;
the composition comprises:
from about 30 to about 50 vol. % bone marrow;
about 0.5 to about 3 vol. % methylene blue; and
about 15 to about 25 vol. % jojoba oil; and
a dried botanical is infused directly into a mixture comprising the bone marrow, the tallow, and the carrier oil.
